(19) United States
(12) Patent Application Publication
(10) Pub. No.: US 2006/0009524 A1
(43) Pub. Date: Jan. 12, 2006

(54) Methods for producing D-beta-hydroxyamino acids

(75) Inventors: Hiroaki Yamamoto, Ibaraki (JP); Takanori Nakajima, Ibaraki (JP); Hiroki Tanaka, Niigata (JP)

Correspondence Address: LAHIVE & COCKFIELD, LLP., 28 STATE STREET, BOSTON, MA, 02109, US

(73) Assignee: Daicel Chemical Industries, Ltd., Osaka (JP)

(21) Appl. No.: 11/165,466

(22) Filed: Jun. 22, 2005

(30) Foreign Application Priority Data
Jun. 25, 2004 (JP) 2004-188583

(51) Int. Cl. A61K 31/195; C07C 227/14

(52) U.S. Cl. 514/567; 562/564

(57) ABSTRACT

An objective of the present invention is to provide efficient methods for producing D-β-hydroxyamino acids (formula 2 or 4), such as D-erythro-2-amino-3-cyclohexyl-3-hydroxypropionic acid, which are useful as intermediates in the synthesis of pharmaceutical products and others.

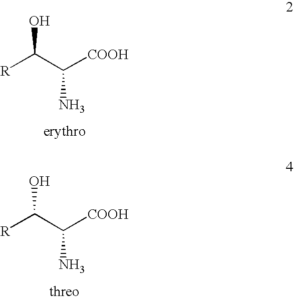

The present invention makes it possible to efficiently produce D-erythro-2-amino-3-cyclohexyl-3-hydroxypropionic acid by cleaving unnecessary L-erythro-2-amino-3-cyclohexyl-3-hydroxypropionic acid in industrially feasible concentrations of DL-erythro-2-amino-3-cyclohexyl-3-hydroxypropionic acid used as starting material by using Pseudomonas putida-derived L-phenylserine aldolase.

### FIELD OF THE INVENTION

The present invention relates to methods for producing D-erythro-β-hydroxyamino acids and D-threo-β-hydroxyamino acids which are useful as intermediates in the synthesis of pharmaceutical products, pesticides, and others.

### BACKGROUND OF THE INVENTION

To date, D-erythro-3-hydroxyamino acids have been produced by the chemical synthesis method as described below. An aldehyde derivative and glycine are condensed together in the presence of a strong alkali to give a mixture of racemic threo/erythro-hydroxyamino acid derivatives. Then, the threo and erythro isomers are separated from each other. A substituent is introduced into the amino moiety of the racemic erythro-hydroxyamino acid obtained. Optical resolution is then carried out using an optical resolving agent, such as quinine and brucine. In the final step, the substituent is removed from the amino moiety to yield the final product. However, this procedure has problems, namely, complicated steps and low yield. In addition, the optical resolving agent used in the procedure is expensive, and thus the procedure entails high cost.

In Unexamined Published Japanese Patent Application No. (JP-A) Hei 1-317391 and JP-A Hei 2-207793, D-β-hydroxyamino acid is produced by reacting glycine or glycine-metal chelate with an aldehyde derivative in the presence of D-threonine aldolase. These methods can specifically produce the D configuration for the α-amino group, but result in both threo and erythro configurations for the -β-hydroxyl group. Thus, the diastereoisomer selectivity is poor in these methods.

JP-A Hei 6-165693 discloses a method for producing D-erythro-β-hydroxyamino acid by reacting racemic erythro-α-hydroxyamino acid with L-allothreonine aldolase, which is an enzyme that specifically cleaves L-erythro-β-hydroxyamino acid into glycine and the corresponding aldehyde derivative. However, the document describes that, in this method, the starting material racemic erythro-α-hydroxyamino acid does not markedly inhibit the reaction at the concentration range of 1-100 mM. It was predicted that the substrate or the reaction product would inhibit the reaction when a larger amount of racemate was used. Furthermore, the enzyme reaction may hardly proceed when the racemate is added beyond its solubility.

Accordingly, the conventional methods are industrially unfeasible.

### SUMMARY OF THE INVENTION

An objective of the present invention is to provide methods by which D-β-hydroxyamino acid can be produced even at higher substrate concentrations. More specifically, an objective of the present invention is to provide methods for producing D-β-hydroxyamino acids, such as D-erythro-2-amino-3-cyclohexyl-3-hydroxypropionic acid and D-threo-2-amino-3-cyclohexyl-3-hydroxypropionic acid.

A study of microorganisms assimilating L-phenylserine has revealed that Pseudomonas putida biovar A24-1 strain isolated from soil produces L-phenylserine aldolase. L-phenylserine aldolase is an enzyme that catalyzes the cleavage of L-phenylserine into benzaldehyde and glycine. The enzyme has been purified and its enzymatic properties have been characterized. Furthermore, the gene encoding the enzyme has been cloned, and its nucleotide sequence and the encoded amino acid sequence have been determined (Vitamin (Japan), 75, 51-61, 2001).

The present inventors discovered that, when L-phenylserine aldolase reacted with DL-erythro-2-amino-3-cyclohexyl-3-hydroxypropionic acid (hereinbelow abbreviated as DL-ACHP), one enantiomer, L-ACHP, contained in the starting material was cleaved to cyclohexyl aldehyde and glycine, but the other enantiomer, D-ACHP, remained unreacted. Accordingly, D-ACHP was yielded with high optical purity. L-phenylserine aldolase was found to have an enzymatic activity and high specificity to D-β-hydroxyamino acids, and, thus, meets the requirements for industrial production. For example, L-phenylserine aldolase retained sufficiently high enzymatic activity even at an exceedingly high concentration (15%) of the starting material. Thus, the present invention provides the methods for producing D-erythro-β-hydroxyamino acid and D-threo-β-hydroxyamino acid described below.

[1] A method for producing D-erythro-β-hydroxyamino acid, which comprises the step of collecting D-erythro-α-hydroxyamino acid represented by formula 2 after DL-erythro-β-hydroxyamino acid represented by formula 1 (where R represents an optionally substituted cyclohexyl group, a phenyl group, an alkyl group, or an allyl group) 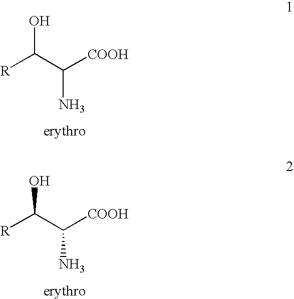
is reacted with at least one enzymatically active material selected from the group consisting of: a protein encoded by any one of the polynucleotides defined in (a) to (e) indicated below, a microorganism or transformant expressing the protein, and a processed product thereof;
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added, further wherein the resulting protein is functionally equivalent to the protein comprising the amino acid sequence of SEQ ID NO: 2;
(d) a polynucleotide hybridizing under stringent conditions to a DNA comprising the nucleotide sequence of SEQ ID NO: 1, wherein said polynucleotide encodes a protein that is functionally equivalent to the protein comprising the amino acid sequence of SEQ ID NO: 2; and
(e) a polynucleotide encoding an amino acid sequence having 70% or higher homology to the amino acid sequence of SEQ ID NO: 2, wherein said polynucleotide encodes a protein that is functionally equivalent to the protein comprising the amino acid sequence of SEQ ID NO: 2.

[2] The method for producing D-erythro-β-hydroxyamino acid according to claim 1, wherein R is an optionally substituted cyclohexyl group.

[3] A method for producing D-threo-β-hydroxyamino acid, which comprises the step of collecting D-threo-β-hydroxyamino acid represented by formula 4 after DL-threo-α-hydroxyamino acid represented by formula 3 (where R represents an optionally substituted cyclohexyl group, a phenyl group, an alkyl group, or an allyl group) 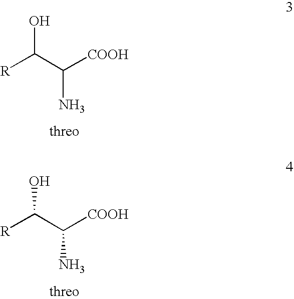
is reacted with at least one enzymatically active material selected from the group consisting of a protein encoded by any one of the polynucleotides defined in (a) to (e) indicated below, a microorganism or transformant expressing the protein, and a processed product thereof,
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added, further wherein the resulting protein is functionally equivalent to the protein comprising the amino acid sequence of SEQ ID NO: 2;
(d) a polynucleotide hybridizing under stringent conditions to a DNA comprising the nucleotide sequence of SEQ ID NO: 1, wherein said polynucleotide encodes a protein that is functionally equivalent to the protein comprising the amino acid sequence of SEQ ID NO: 2; and
(e) a polynucleotide encoding an amino acid sequence having 70% or higher homology to the amino acid sequence of SEQ ID NO: 2, wherein said polynucleotide encodes a protein that is functionally equivalent to the protein comprising the amino acid sequence of SEQ ID NO: 2.

[4] The method for producing D-threo-β-hydroxyamino acid according to claim 3, wherein R is an optionally substituted cyclohexyl group.

[5] The method for producing D-β-hydroxyamino acid according to claim 1 or 3, wherein the concentration of material DL-β-hydroxyamino acid is 30 g/l or higher in the reaction solution.

[6] The method for producing D-β-hydroxyamino acid according to claim 1 or 3, wherein the concentration of material DL-β-hydroxyamino acid is 50 g/l or higher in the reaction solution.

[7] The method for producing D-erythro-α-hydroxyamino acid or D-threo-β-hydroxyamino acid according to claim 1 or 3, wherein the concentration of material DL-erythro-β-hydroxyamino acid or DL-threo-α-hydroxyamino acid is 50 g/l or higher in the reaction solution.

[8] The method for producing D-erythro-β-hydroxyamino acid or D-threo-β-hydroxyamino acid according to claim 2 or 4, which additionally comprises the steps of:
(1) dissolving D-erythro-β-hydroxyamino acid or D-threo-β-hydroxyamino acid by adjusting the pH of the reaction solution to 10 or higher after the reaction;
(2) separating insoluble materials, and
(3) collecting D-erythro-β-hydroxyamino acid or D-threo-β-hydroxyamino acid precipitated by adjusting the pH of the reaction solution to 2 to 9.5.

[9] The method for producing D-erythro-β-hydroxyamino acid or D-threo-β-hydroxyamino acid according to claim 2 or 4, which additionally comprises the steps of:
(1) dissolving D-erythro-β-hydroxyamino acid or D-threo-β-hydroxyamino acid by adjusting the pH of the reaction solution to 1.5 or lower after the reaction;
(2) separating insoluble materials, and
(3) collecting D-erythro-β-hydroxyamino acid or D-threo-β-hydroxyamino acid precipitated by adjusting the pH to 2 to 9.5.

L-phenylserine aldolase has been revealed to have the substrate specificity described below. First, this enzyme efficiently acts on L isomers of the following compounds comprising the aromatic substituents.
DL-threo-phenyl serine;
DL-erythro-phenyl serine; and
DL-thienyl serine; etc.

However, L-phenylserine aldolase has low enzymatic activity to L-threonine and L-allo-threonine. In addition, L-phenylserine aldolase exhibits almost no activity to L-serine. In other words, L-phenylserine aldolase only exhibits strong enzymatic activity to aromatic amino acids. Therefore, it could not have been predicted that L-phenylserine aldolase would have high enzymatic activity to L-ACHP, a compound which contains no aromatic ring.

In general, aldehydes inhibit the aldolase reaction. Namely, cyclohexyl aldehyde generated in the enzymatic decomposition of D-ACHP might inhibit the enzymatic activity of L-phenylserine aldolase. It is predicted that the higher the substrate concentration, the higher the degree of inhibition. Surprisingly, in fact, L-ACHP was decomposed with high efficiency even at exceedingly high substrate concentrations (15% or higher). The enzyme is not activated by monovalent or divalent cations, such as potassium ion, ammonium ion, and manganese ion. This is also different from the features of common threonine aldolases.

In addition, it would have been difficult to predict from its structural features that L-phenylserine aldolase has high enzymatic activity to L-ACHP. For example, JP-A Hei 6-165693 indicated above disclosed the nucleotide and amino acid sequences of L-allothreonine aldolase derived from Aeromonas jandaei, which is one of L-allothreonine aldolases used in the production of D-erythro-α-hydroxyamino acid. When its amino acid sequence was compared with that of L-phenylserine aldolase, the homology between the two was 20.4%. Thus, there is a great structural difference between them.

The L-phenylserine aldolase used in the present invention is an enzyme classified under EC 4.1.2.26, for which L-threo isomer and L-erythro isomer of 3-phenyl serine are specific substrates. On the other hand, L-allothreonine aldolase is classified under EC 2.1.2.1 and exhibits the strongest activity to L-allothreonine. Thus, the two are unrelated enzymes in terms of biochemistry and enzyme taxonomy.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### DETAILED DESCRIPTION OF THE INVENTION

The words “a”, “an”, and “the” as used herein mean “at least one” unless otherwise specifically indicated.

Unless otherwise defined, all technical and scientific terms used herein have the same meaning as commonly understood by one of ordinary skill in the art to which this invention belongs. In case of conflict, the present specification, including definitions, will control.

The present invention is directed to methods for producing D-erythro-β-hydroxyamino acid, which comprise the steps of reacting an enzymatically active material having L-phenylserine aldolase activity with DL-erythro-β-hydroxyamino acid represented by formula 1 indicated above and collecting the remaining D-erythro-β-hydroxyamino acid represented by formula 2 indicated above. The present invention is also directed to methods for producing D-threo-α-hydroxyamino acid, which comprise the steps of reacting an enzymatically active material having L-phenylserine aldolase activity with DL-threo-α-hydroxyamino acid represented by formula 3 indicated above and collecting remaining D-threo-β-hydroxyamino acid represented by formula 4 indicated above.

DL-erythro-β-hydroxyamino acid and DL-threo-β-hydroxyamino acid used as the starting materials in the methods for producing D-β-hydroxyamino acid according to the present invention have the structures represented by formulae 1 and 3 below. Herein, each compound refers to either or both of erythro isomer and threo isomer unless otherwise specified. For example, “D-β-hydroxyamino acid” refers to “either or both of D-erythro-β-hydroxyamino acid and D-threo-β-hydroxyamino acid”.

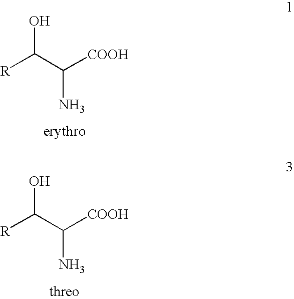

The R group in formula 1 or 3 is, for example, a cyclohexyl group, a phenyl group, an alkyl group, or an allyl group, which may or may not be substituted with a lower alkyl group, a halogen group, a nitro group, an alkoxy group, a hydroxyl group, or such. More specifically, the R group includes, for example, a cyclohexyl group, a phenyl group, and a thienyl group.

DL-erythro-β-hydroxyamino acid represented by formula 1 can be synthesized, for example, by decyclizing trans-2,3-epoxy carboxylic acid by reacting it with an amine, such as ammonia and benzylamine, and, if required, deprotecting it, as shown in formula 5.

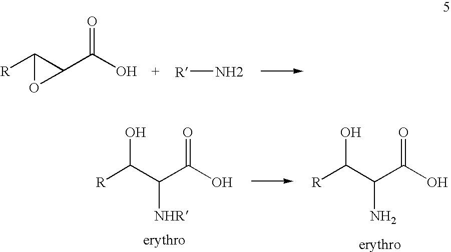

Furthermore, DL-threo-β-hydroxyamino acid represented by formula 3 can be synthesized, for example, threo-selectively by aldol condensation between aldehyde and glycine as shown in formula 6. The compound can also be synthesized by decyclizing cis-2,3-epoxy carboxylic acid using an amine, such as ammonia and benzylamine, and, if required, deprotecting it.

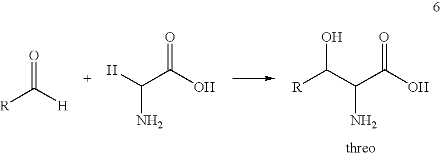

When the R group in formula 1 or 3 is a cyclohexyl group, the compound can be synthesized by preparing-β-hydroxyamino acid represented by formula 1 or 3 where the R group is a phenyl group and then converting the phenyl group to a cyclohexyl group via reduction (addition of hydrogen).

In preferred embodiments of the present invention, D-erythro-2-amino-3-hydroxypropionic acid is produced by reacting the enzymatically active material with the starting material DL-erythro-2-amino-3-hydroxypropionic acid. The enzymatically active material is allowed to react with DL-β-hydroxyamino acid under conditions preferable to maintain its enzymatic activity.

First, there is no limitation on the concentration of the starting material DL-β-hydroxyamino acid in the reaction. The concentration is typically about 0.1 to about 30%, preferably 0.5 to 20%, more preferably 1 to 15%. DL-erythro-α-hydroxyamino acid used as the starting material in the present invention is a mixture of D-erythro-β-hydroxyamino acid and L-erythro-α-hydroxyamino acid. The ratio between D and L isomers in the mixture ranges from 10:90 to 90:10, preferably 25:75 to 75:25, more preferably 50:50 (racemate). Likewise, DL-threo-β-hydroxyamino acid used as the starting material is a mixture of D-threo-β-hydroxyamino acid and L-threo-α-hydroxyamino acid, and the ratio between D and L isomers in the mixture ranges from 10:90 to 90:10, preferably 25:75 to 75:25, more preferably 50:50 (racemate).

Specifically, the concentration of the starting material DL-β-hydroxyamino acid in the reaction solution may be, for example, 30 g/l or higher, or 50 g/l. More specifically, the starting material can be added at a concentration of 30-200 g/l or 30-150 g/l, for example, 60-100 g/l. The starting material can be added all at once at the start of reaction, or alternatively, continuously or stepwise to the reaction solution.

Herein, the phrase “concentration of the starting material” refers to the percentage of the starting material in the reaction solution. The starting material is not necessarily completely dissolved. Namely, in the present invention, the concentration depends on the volume of the reaction solution and the weight of the starting material in the reaction solution (the amount added) regardless of the dissolved state of the starting material. When the starting material is not completely dissolved, the starting material is considered to be saturated in the liquid phase of the reaction solution.

In the present invention, when at least some of the starting material is dissolved in the reaction solution, the necessary reactions will proceed. The L isomer in the dissolved starting material containing both D and L isomers is cleaved to glycine and the corresponding aldehyde by the enzymatically active material. As a result, the concentration of the starting material dissolved in the reaction solution is decreased and thus the starting material is newly dissolved. The L isomer dissolved in the solution is cleaved successively. On the other hand, the D isomer saturated is crystallized in the solution. As a result of the successive consumption of the L isomer in the reaction, the D isomer of interest can be yielded efficiently even when the solubility of starting material is low. For example, under standard conditions, only a small amount of DL-erythro-2-amino-3-cyclohexyl-3-hydroxypropionic acid or DL-threo-2-amino-3-cyclohexyl-3-hydroxypropionic acid, which is used as the starting material in the present invention, is dissolved in the solvent constituting the reaction solution. However, based on the mechanism described above, the D isomer of interest can be collected efficiently according to the present invention.

Herein, the unit “%” refers to “weight/volume (w/v)”. The unit “e.e.” is defined by the following formula for D-erythro-β-hydroxyamino acid:
(([concentration of D-erythro isomer]+[concentration of L-erythro isomer]) /([concentration of D-erythro isomer]+[concentration of L-erythro isomer]))×100

Likewise, the unit “e.e.” is defined by the following formula for D-threo-β-hydroxyamino acid:
(([concentration of D-threo isomer]−[concentration of L-threo isomer]) /([concentration of D-threo isomer]+[concentration of L-threo isomer]))×100

L-phenylserine aldolase is used typically at a concentration of about 0.01 to 10,000 U/ml, preferably at about 0.1 to 1000 U/ml, more preferably at about 1 to 500 U/ml. The reaction temperature may be selected from a range wherein the enzymatic activity of the enzymatically active material can be maintained. Specifically, the reaction temperature is typically 5-60° C., preferably 10-50° C., more preferably 20-40° C. Furthermore, the pH of reaction may also be selected from a range wherein the enzymatic activity of the enzymatically active material can be maintained. The pH typically ranges from 6 to 11, preferably from 7 to 10, more preferably from 8 to 9.5. The reaction can be performed with or without agitation.

In the present invention, additives may be added optionally to the reaction solution containing the enzymatically active material and the starting material. Such an additive is added to enhance or stabilize the enzymatic activity, to increase the fluidity of the reaction solution, or for other purposes. L-phenylserine aldolase used in the present invention uses pyridoxal-5′-phosphate (PLP) as the co-enzyme. Thus, the enzymatic activity can be enhanced and stabilized by adding PLP to the reaction solution. The concentration of PLP in the reaction solution typically ranges from 0.0001 to 10 mM, preferably from 0.001 to 1 mM, more preferably from 0.005 to 0.1 mM. Furthermore, the fluidity of the reaction solution is sometimes improved by adding Tris-hydrochloride buffer, potassium phosphate buffer, or others to the reaction solution.

The production of D-β-hydroxyamino acid according to the present invention can be performed using an aqueous solvent, an organic solvent which is immiscible with water, or a mixed solvent comprising two phases of aqueous solvent and water-insoluble organic solvent. Examples of water-insoluble organic solvents which can be used in the present invention include, but are not limited to, ethyl acetate, butyl acetate, toluene, chloroform, n-hexane, methyl isobutyl ketone, methyl t-butyl ether, and diisopropyl ether. On the other hand, the aqueous solvents which can be used in the present invention include, for example, water and buffers that maintain the enzymatic activity of the enzymatically active material.

Furthermore, the starting material and the enzymatically active material can also be contacted in a water-soluble organic solvent or in a mixed solvent of an aqueous solvent and a water-soluble organic solvent. Examples of water-soluble organic solvents include, but are not limited to, methanol, ethanol, isopropyl alcohol, acetonitrile, acetone, and dimethylsulfoxide. The reaction of the present invention can be conducted using immobilized enzyme, membrane reactor, or the like.

In the present invention, L-β-hydroxyamino acid in the starting material is consumed by reacting the starting material with the enzymatically active material, and D-β-hydroxyamino acid, which is the compound of interest, remains intact in the reaction system. Herein, when a stereospecific compound of interest remains intact, it is sometimes stated that the compound is produced. In the present invention, the L isomer is removed enzymatically from the starting material containing both D and L isomers, and the remaining D isomer is collected as the compound of interest. The D isomer itself is originally contained in the starting material. However, the optical purity of the D isomer is increased from a lower state with the coexisting L isomer, which means that the D isomer is generated.

D-β-hydroxyamino acid remaining intact after reaction can be purified by appropriately using in combination solubilization; separation by centrifugation, filtration, or the like; extraction with organic solvents; various chromatographic methods, such as ion-exchange chromatography; adsorption using adsorbing agents; dehydration or flocculation using dehydrating agent or flocculating agent; crystallization; distillation; etc. D-β-hydroxyamino acid can be solubilized by alkalization or acidification.

For example, the solubility of D-erythro-2-amino-3-cyclohexyl-3-hydroxypropionic acid in water is lower, and thus most of it is precipitated at pHs where L-phenylserine aldolase of the present invention is active. D-erythro-2-amino-3-cyclohexyl-3-hydroxypropionic acid remaining in the reaction solution can be dissolved by adjusting the pH of the solution to 1.5 or lower with an acid, such as hydrochloric acid, sulfuric acid, and nitric acid, after reaction. Alternatively, D-erythro-2-amino-3-cyclohexyl-3-hydroxypropionic acid can be dissolved by adjusting the pH of the reaction solution to 10 or higher with an alkaline, such as sodium hydroxide and potassium hydroxide.

The reacted product is dissolved, and then, if required, a flocculating agent may be added. Then, microbes and proteins can be removed by centrifugation or filtration. Aldehyde generated in the reaction can be removed, for example, by extracting it using an organic solvent in which the solubility of aldehyde is higher but the solubility of D-erythro-2-amino-3-cyclohexyl-3-hydroxypropionic acid is lower. Such organic solvents used to remove aldehyde in the present invention include, but are not limited to, xylene, hexane, toluene, t-methyl butyl ether, methyl isobutyl ketone, ethyl acetate, and butyl acetate. Aldehyde recovered by extraction with the organic solvent may be recycled. After organic solvent extraction, D-erythro-2-amino-3-cyclohexyl-3-hydroxypropionic acid can be recovered from the aqueous phase by known methods, such as recrystallization using concentration or isoelectric precipitation, treatment with ion-exchange resins, and membrane separation.

On the other hand, the enzymatically active material according to the present invention can be prepared, for example, using a microorganism capable of producing L-phenylserine aldolase characterized by the properties described below in (1)-(3), which belongs to Pseudomonas putida. The microorganism belonging to Pseudomonas putida preferably include, for example, Pseudomonas putida biovar A24-1 strain. Namely, L-phenylserine aldolase characterized by the properties described below and isolated from a microorganism belonging to Pseudomonas putida can be used as the enzymatically active material of the present invention. Alternatively, a microorganism belonging to Pseudomonas putida, which produces the enzyme, or a processed product of the microorganism, can be used as the enzymatically active material of the present invention.

(1) Activity:

catalyzes the cleavage of L-phenylserine into benzaldehyde and glycine.
   (2) Substrate Specificity:
(a) acts on both L-threo-phenyl serine and L-erythro-phenyl serine, but has substantially no activity to D-threo-phenyl serine and D-erythro-phenyl serine.
(b) acts on the L-erythro isomer of DL-erythro-2-amino-3-cyclohexyl-3-hydroxypropionic acid, but has substantially no activity to the D-erythro isomer.
   (3) Molecular Weight:
190,000 to 210,000 in gel filtration and 35,000 in sodium dodecyl sulfate-polyacrylamide gel electrophoresis.

The activity of L-phenylserine aldolase of the present invention can be tested, for example, by the procedure described below.

Assay Method for the Activity:

0.5 ml of the reaction solution containing 20 mM DL-threo-phenyl serine, 200 mM TAPS-NaOH buffer (pH 8.5), 20 μM pyridoxal-5′-phosphate (hereinafter abbreviated as PLP), and the enzyme is incubated at 30° C. for 10 minutes. The reaction is terminated by adding 0.5 ml of 1N HCl. Benzaldehyde generated is quantified by the method described below. A solution of 2N hydrochloric acid containing 0.15 ml of 0.1% 2,4-dinitrophenyl hydrazine is added to 1.0 ml of the solution after the reaction. The mixture is stirred quickly, and allowed to stand still at 30° C. for 20 minutes. Then, 3 ml of ethanol is added and the resulting mixture is stirred quickly. 0.85 ml of 3N NaOH is added and the resulting mixture is allowed to stand still for 10 minutes. The absorbance of the solution is determined at 475 nm. The amount of enzyme that catalyzes the generation of 1 μmol benzaldehyde at 30° C. for 1 minute is defined as 1 unit (U) of enzyme activity.

Herein, the phrase “an enzyme has substantially no activity to a compound” means that, when each compound is given as a substrate in the assay method for the activity described above and the activity to a preferred substrate determined under the same conditions is taken as 100, the activity to the compound is, for example, 10% or lower, preferably 5% or lower. Specifically, the activities of L-phenylserine aldolase derived from Pseudomonas putida biovar A24-1 strain to the compounds indicated below, which are relative to corresponding preferred substrates, are as follows:
(a) When the activity to L-threo-phenyl serine or L-erythro-phenyl serine is taken as 100, the activities to D-threo-phenyl serine and D-erythro-phenyl serine are undetectable.
(b) When the activity to the L-erythro isomer of DL-erythro-2-amino-3-cyclohexyl-3-hydroxypropionic acid is taken as 100, the activity to the D-erythro isomer is undetectable.

Pseudomonas putida biovar A24-1 strain can be cultured using conventional media for bacterial culture. The enzyme is induced by DL-threo-phenyl serine or such. Thus, it is preferred to add an inducer to the medium. The culture medium preferably used is, for example, a peptone medium (pH 7.2) containing 1.0% peptone, 0.2% dipotassium monohydrogen phosphate, 0.2% monopotassium dihydrogen phosphate, 0.2% sodium chloride, 0.01% magnesium sulfate heptahydrate, and 0.01% yeast extract, supplemented with 0.2% DL-threo-phenyl serine.

L-phenylserine aldolase can be purified from culture of a microorganism, for example, by the procedure described below. Pseudomonas putida biovar A24-1 strain is expanded sufficiently in the peptone medium described above containing 0.2% DL-threo-phenyl serine. Then, the bacterial cells are harvested and lysed in a buffer to give cell-free extract. In this treatment, it is preferred to add the agents indicated below to the buffer to protect the enzyme.
Reducing agent: 2-mercaptoethanol or the like
Protease inhibitor: phenylmethanesulfonyl fluoride, pepstatin A, leupeptin, metal chelating agent, etc.
PLP

From the cell-free extract thus obtained, the enzyme of interest can be purified, for example, by appropriately using in combination various protein fractionation methods and chromatographic methods listed below.
Fractionation methods based on protein solubility (precipitation using organic solvents, salting out with ammonium sulfate, etc.)
Cation-exchange chromatography, anion-exchange chromatography, gel filtration, and hydrophobic chromatography
Affinity chromatography using chelating agents, dyes, antibodies, and others

More specifically, the enzyme of interest can be purified from the cell-free extract until it gives a single band in electrophoresis, for example, according to the respective steps described below. For example, detailed experimental conditions of the respective steps may be the same as described in Examples.
Fractionation with 40-60% ammonium sulfate;
DEAE-cellulose ion-exchange chromatography;
Hydroxyapatite chromatography;
DEAE-cellulose anion-exchange chromatography (2nd);
Hydroxyapatite chromatography (2nd);
MonoQ anion-exchange chromatography.

L-phenylserine aldolase derived from Pseudomonas putida biovar A24-1 strain is a preferred enzymatically active material to be used in the present invention. The enzyme comprises the amino acid sequence of SEQ ID NO: 2. The enzymatically active material to be used in the present invention also includes homologues of the protein comprising the amino acid sequence of SEQ ID NO: 2. In other words, the enzymatically active material having L-phenylserine aldolase activity according to the present invention includes at least one enzymatically active material selected from the group consisting of the protein encoded by any one of the polynucleotides defined in (a) to (e) indicated below, a microorganism or transformant expressing the protein, and a processed product thereof.
(a) a polynucleotide comprising the nucleotide sequence of SEQ ID NO: 1;
(b) a polynucleotide encoding a protein comprising the amino acid sequence of SEQ ID NO:2;
(c) a polynucleotide encoding a protein comprising the amino acid sequence of SEQ ID NO: 2, in which one or more amino acids have been substituted, deleted, inserted, and/or added;
(d) a polynucleotide hybridizing under stringent conditions to a DNA comprising the nucleotide sequence of SEQ ID NO: 1; and
(e) a polynucleotide encoding an amino acid sequence having 70% or higher homology to the amino acid sequence of SEQ ID NO: 2.

Furthermore, proteins comprising the amino acid sequence of SEQ ID NO: 2 which contains additional amino acid sequences can also be used as the enzymatically active materials in the present invention, so long as they have the same activity as that of the protein comprising the amino acid sequence of SEQ ID NO: 2 (i.e., catalyze the cleavage of L-phenylserine into benzaldehyde and glycine). For example, proteins comprising the amino acid sequence of SEQ ID NO: 2 additionally containing one or more His tags and such are included in the enzymatically active material in the present invention. In addition, transformants expressing such proteins and recombinants produced by them are also included in the enzymatically active material in the present invention.

The homologue of L-phenylserine aldolase to be used in the present invention refers to a protein comprising the amino acid sequence of SEQ ID NO: 2 in which one or more amino acids have been deleted, substituted, inserted, and/or added and which is functionally equivalent to the protein comprising the amino acid sequence of SEQ ID NO: 2. Herein, the phrase “a protein functionally equivalent to the protein comprising the amino acid sequence of SEQ ID NO: 2” refers to a protein having physicochemical and enzymatic properties described above in (1) to (3).

Those skilled in the art can prepare a polynucleotide encoding the homologue of L-phenylserine aldolase by introducing appropriate substitutions, deletions, insertions, and/or additions into the DNA of SEQ ID NO: 1 using site-directed mutagenesis (Nucleic Acid Res. 10, pp. 6487 (1982); Methods in Enzymol. 100, pp. 448 (1983); Molecular Cloning 2nd Ed., Cold Spring Harbor Laboratory Press (1989); PCR A Practical Approach IRL Press pp. 200 (1991)) or the like. The homologue of L-phenylserine aldolase of SEQ ID NO: 2 can be obtained by introducing and expressing the polynucleotide encoding the homologue of L-phenylserine aldolase in a host.

In the amino acid sequence of SEQ ID NO: 2, the acceptable number of mutations are, for example, 100 amino acid residues or less, typically 50 amino acid residues or less, preferably 30 amino acid residues or less, more preferably 15 amino acid residues or less, still more preferably 10 amino acid residues or less, or 5 amino acid residues or less. In general, it is preferred to select an amino acid having characteristics similar to those of the original amino acid before substitution to retain the protein function. Such amino acid substitution is called “conservative substitution”. For example, Ala, Val, Leu, Ile, Pro, Met, Phe, and Trp are categorized into the group of non-polar amino acids and have similar characteristics. The group of uncharged amino acids includes Gly, Ser, Thr, Cys, Tyr, Asn, and Gln. The group of acidic amino acids includes Asp and Glu. The group of basic amino acids includes Lys, Arg, and His. The amino acid substitution between members within the same group is preferable.

Furthermore, the homologue of the polynucleotide in the present invention includes polynucleotides capable of hybridizing under stringent conditions to a polynucleotide comprising the nucleotide sequence of SEQ ID NO: 1, which encode proteins having the physicochemical properties of (1) and (2) described above. The phrase “a polynucleotide capable of hybridizing under stringent conditions” refers to a polynucleotide hybridizing to probe DNA selected from one or more of sequences comprising at least any 20 consecutive residues, preferably at least any 30 consecutive residues, for example, any 40, 60, or 100 consecutive residues from the sequence of SEQ ID NO: 1. The hybridization is carried out, for example, using ECL direct nucleic acid labeling and detection system (Amersham Pharmacia Biotech) under the conditions described in the attached manual (for example, washing at 42° C. with primary wash buffer containing 0.5×SSC). More specifically, the term “stringent conditions” includes but is not limited to, for example, the typical conditions comprising 42° C., 2×SSC, and 0.1% SDS, preferably 50° C., 2×SSC, and 0.1% SDS, and more preferably 65° C., 0.1×SSC, and 0.1% SDS. There are various factors influencing the hybridization stringency, which include temperature and salt concentration. Those skilled in the art can select such factors appropriately for optimal stringency.

The phrase “homologue of L-phenylserine aldolase of the present invention” refers to a protein exhibiting at least 70% homology, preferably at least 80% homology, more preferably 90%, even more preferably 95% or higher homology to the amino acid sequence of SEQ ID NO: 2. Protein homology searches can be carried out, for example, by searching amino acid sequence databases for proteins, such as SWISS-PROT, PIR, and DAD, DNA sequence databases, such as DDBJ, EMBL, and GenBank, and databases of predicted amino acid sequences based on DNA sequences, for example, via Internet, using programs, such as BLAST and FASTA.

Blast homology searches against the amino acid sequence of SEQ ID NO: 2 revealed that a putative low-substrate-specificity aldolase (68%) derived from Ralstonia solanacearum exhibited the highest homology to the sequence. Of proteins whose functions have been identified, the low-substrate-specificity L-threonine aldolase derived from Pseudomonas aeruginosa PAO1 strain showed 41% homology to the sequence of SEQ ID NO: 2 at the amino acid level.

A DNA of interest can be yielded by screening DNAs obtained directly from environmental samples, such as soil, using a polynucleotide encoding L-phenylserine aldolase as a probe. DNA libraries to be used in such screening can be obtained by introducing DNA digests, which have been prepared by shearing or enzymatic treatment of DNAs obtained from environmental samples, into phage, plasmid, or the like, and transforming E. coli with the construct. Such screening can be carried out by using colony or plaque hybridization. After hybridizing DNAs are obtained by the method described above, the nucleotide sequences of the DNAs are determined. When the encoded amino acid sequence has 70% or higher homology to the sequence of L-phenylserine aldolase, the protein is expected to have a function similar to that of the aldolase. E. coli transformed with a plasmid containing such a DNA can also be used in the present invention.

A polynucleotide encoding L-phenylserine aldolase can be isolated, for example, by the method described below. For example, a DNA of interest can be obtained by carrying out PCR using as a template chromosomal DNA or cDNA library from a strain producing the enzyme and PCR primers designed based on the nucleotide sequence of SEQ ID NO: 1.

Furthermore, such a DNA of interest can be obtained by screening a library from the strain producing the enzyme using as a probe a DNA fragment obtained by PCR. Such libraries include cDNA libraries and libraries prepared by introducing restriction enzyme-treated chromosomal DNA into phage, plasmid, or the like, and transforming E. coli with the construct. Such screening can be carried out by using colony or plaque hybridization.

The nucleotide sequence of a DNA fragment obtained by PCR can be used to obtain its 5′- and 3′-side nucleotide sequences. This can be achieved by RACE (Rapid Amplification of cDNA End, “Experimental Manuel for PCR” p. 25-33, HBJ Publisher). In addition, inverse PCR (Genetics 120, 621-623, 1988) has been known as a method of identifying unknown sequences based on known fragment sequence information. Inverse PCR uses DNA libraries in which DNAs are self-circularized. Such a library can be prepared by digesting chromosomal DNA from a strain producing the enzyme with an appropriate restriction enzyme and self-circularizing the DNA. On the other hand, primers for inverse PCR are designed so that the synthesis of complementary strand proceeds outside the nucleotide sequence of a known cDNA (unknown regions). Since the template DNA is circular, a sequence segment covering an unknown region can be obtained as an amplified product by inverse PCR.

The polynucleotides of the present invention include not only genomic DNAs and cDNAs cloned by the method described above but also synthetic DNAs.

The polynucleotides encoding a homologue isolated by the method described above can be inserted into a known expression vector to give an expression vector for the homologue of L-phenylserine aldolase. The recombinant protein for the homologue of L-phenylserine aldolase can be prepared by culturing transformants containing the expression vector. Such transformants thus prepared, and recombinant homologues produced by them, are included in the enzymatically active materials in the present invention.

In the present invention, there is no limitation on the type of microorganism to be transformed to express L-phenylserine aldolase or a homologue thereof, so long as it can be transformed with a recombinant vector comprising a polynucleotide encoding a polypeptide having the L-phenylserine aldolase activity and express the L-phenylserine aldolase activity. Such microorganisms include, for example, the microorganisms listed below.
Bacteria for which host-vector systems are developed:
   the genus Escherichia,
   the genus Bacillus,
   the genus Pseudomonas,
   the genus Serratia,
   the genus Brevibacterium,
   the genus Corynebacterium,
   the genus Streptococcus, or
   the genus Lactobacillus.
Actinomycetes for which host-vector systems are developed:
   the genus Rhodococcus or
   the genus Streptomyces.
Yeast for which host-vector systems are developed:
   the genus Saccharomyces,
   the genus Kluyveromyces,
   the genus Schizosaccharomyces,
   the genus Zygosaccharomyces,
   the genus Yarrowia,
   the genus Trichosporon,
   the genus Rhodosporidium,
   the genus Pichia, or
   the genus Candida.
Fungi for which host-vector systems are developed:
   the genus Neurospora,
   the genus Aspergillus,
   the genus Cephalosporium, or
   the genus Trichoderma.

The procedure for generating transformants and constructing recombinant vectors suitable for hosts can be performed according to standard techniques known in the fields of molecular biology, bioengineering, and genetic engineering (for example, Sambrook et al., Molecular Cloning, Cold Spring Harbor Laboratories).

To express an L-phenylserine aldolase gene of the present invention in microbial cells and such, first, a polynucleotide of the present invention may be inserted into a plasmid vector or a phase vector stably existing in the microorganisms, and the genetic information is transcribed and translated. In addition, a promoter, which regulates transcription and translation, may be inserted 5′-upstream of the polynucleotide of the present invention; preferably, a terminator is also inserted 3′-downstream of the polynucleotide. The promoter and terminator should function in microorganisms to be used as host cells. Vectors, promoters, and terminators functioning in various microorganisms are described in, for example, “Biseibutsugaku Kisokouza” (Basic Course of Microbiology) Vol. 8 Idenshikougaku (Genetic Engineering), Kyoritsu Shuppan Co., Ltd., particularly for yeast, described in “Adv. Biochem. Eng. 43, 75-102 (1990), Yeast 8, 423-488 (1992)”, etc.

For example, plasmid vectors such as pBR and pUC series, and promoters such as those of β-galactosidase (lac), tryptophan operon (trp), tac, trc (fusion of lac and trp), and those derived from λ-phage PL, PR, etc. can be used for the genus Escherichia, particularly Escherichia coli. Terminators derived from trpA, phage, and rmB ribosomal RNA can also be used.

Vectors such as the pUB 110 and pC194 series can be used for the genus Bacillus and can be integrated into chromosomes. Promoters and terminators such as those of alkaline protease (apr), neutral protease (npr), and amy α-amylase) can be used.

Host-vector systems for the genus Pseudomonas, specifically Pseudomonas putida and Pseudomonas cepacia, have been developed. A broad host range vector pKT240 (containing genes necessary for autonomous replication derived from RSF1010) based on plasmid TOL that is involved in degradation of toluene compounds can be utilized. A promoter and terminator of a lipase (JP-A Hei 5-284973) gene and the like can be used.

Plasmid vectors such as pAJ43 (Gene 39, 281 (1985)) can be used for the genus Brevibacterium, especially Brevibacterium lactofermentum. Promoters and terminators for the genus Escherichia can be used for this microorganism.

Plasmid vectors such as pCS11 (JP-A Sho 57-183799) and pCB101 (Mol. Gen. Genet. 196, 175 (1984)) can be used for the genus Corynebacterium, particularly, Corynebacterium glutamicum.

Plasmid vectors such as pHV1301 (FEMS Microbiol. Lett., 26, 239 (1985)) and pGK1 (Appl. Environ. Microbiol. 50, 94 (1985)) can be used for the genus Streptococcus.

For the genus Lactobacillus, pAMβ1 developed for the genus Streptococcus (J. Bacteriol. 137, 614 (1979)) can be used, and some of the promoters for the genus Escherichia are applicable.

For the genus Rhodococcus, a plasmid vector isolated from Rhodococcus rhodochrous and such can be used (J. Gen. Microbiol. 138, 1003 (1992)).

Plasmids functioning in the genus Streptomyces can be constructed by the method described in “Genetic Manipulation of Streptomyces: A Laboratory Manual Cold Spring Harbor Laboratories by Hopwood et al. (1985).” For example, pIJ486 (Mol. Gen. Genet. 203, 468-478 (1986)), pKC1064 (Gene 103, 97-99 (1991)), and pUWL-KS (Gene 165, 149-150 (1995)) can be used, particularly for Streptomyces lividans. Such plasmids can also be used for Streptomyces virginiae (Actinomycetol. 11, 46-53 (1997)).

Plasmids such as the YRp, YEp, YCp, and YIp series can be used for the genus Saccharomyces, especially for Saccharomyces cerevisiae. Integration vectors (such as EP 537456) using homologous recombination with multiple copies of a ribosomal DNA in genomic DNA are extremely useful because they are capable of introducing multiple copies of genes into the host genome and stably maintaining the genes. Furthermore, promoters and terminators of alcohol dehydrogenase (ADH), glyceraldehyde-3-phosphate dehydrogenase (GAPDH), acid phosphatase (PHO), β-galactosidase (GAL), phosphoglycerate kinase (PGK), enolase (ENO), etc. can be used.

Plasmids such as the series of 2 μm plasmids derived from Saccharomyces cerevisiae, the series of pKD1 plasmids (J. Bacteriol. 145, 382-390 (1981)), plasmids derived from pGK11 involved in killer activity, the series of KARS plasmids containing an autonomous replication gene from the genus Kluyveromyces, and vector plasmids (such as EP 537456) capable of being integrated into chromosomes by homologous recombination with ribosomal DNA can be used for the genus Kluyveromyces, particularly for Kluyveromyces lactis. Promoters and terminators derived from ADH and PGK are applicable.

For the genus Schizosaccharomyces, plasmid vectors containing ARS (a gene involved in autonomous replication) derived from Schizosaccharomyces pombe and containing selective markers supplementing auxotrophy of Saccharomyces cerevisiae can be used (Mol. Cell. Biol. 6, 80 (1986)). Furthermore, ADH promoter derived from Schizosaccharomyces pombe is applicable (EMBO J. 6, 729 (1987)). In particular, pAUR224 is commercially available from Takara Shuzo.

For the genus Zygosaccharomyces, plasmid vectors such as pSB3 (Nucleic Acids Res. 13, 4267 (1985)) derived from Zygosaccharomyces rouxii can be used. Promoters of PHO5 derived from Saccharomyces cerevisiae and glycerolaldehyde-3-phosphate dehydrogenase (GAP-Zr) derived from Zygosaccharomyces rouxii (Agri. Biol. Chem. 54, 2521 (1990)), etc. are available.

A host-vector system has been developed for Pichia angusta (previous name: Hansenula polymorpha) among the genus Pichia. Usable vectors include Pichia angusta-derived genes (HARS 1 and HARS2) involved in autonomous replication, but they are relatively unstable. Therefore, multi-copy integration of the gene into a chromosome is effective (Yeast 7, 431-443 (1991)). Promoters of AOX (alcohol oxidase) and FDH (formate dehydrogenase), which are induced by methanol and such, are also available. Host-vector systems for Pichia pastoris have been developed using genes such as PARS1 and PARS2 involved in autonomous replication derived from Pichia (Mol. Cell. Biol. 5, 3376 (1985)). Promoters, such as a promoter of AOX with strong promoter activity induced by high-density culture and methanol, are applicable (Nucleic Acids Res. 15, 3859 (1987)).

For the genus Candida, host-vector systems have been developed for Candida maltosa, Candida albicans, Candida tropicalis, Candida utilis, etc. Vectors for Candida maltosa using ARS, which was cloned from this strain, have been developed (Agri. Biol. Chem. 51, 51, 1587 (1987). Strong promoters for vectors that are able to be integrated into chromosomes have been developed for Candida utilis (JP-A Hei 08-173170).

In the genus Aspergillus, Aspergillus niger and Aspergillus oryzae have been most extensively studied. Plasmids able to be integrated into chromosomes are available. Promoters derived from extracellular protease and amylase are available (Trends in Biotechnology 7, 283-287 (1989)).

For the genus Trichoderma, host-vector systems based on Trichoderma reesei have been developed, and promoters derived from extracellular cellulase genes are available (Biotechnology 7, 596-603 (1989)).

Various host-vector systems for not only microorganisms but also plants and animals have been developed previously. Specifically, such systems to express foreign proteins on a large scale in insects such as silkworm (Nature 315, 592-594, 1985) or plants such as cole, maize, and potato have been developed previously. Such host-vector systems can be used in the present invention.

A method of the present invention comprises the steps of reacting a starting material with the above-mentioned enzymatically active material having L-phenylserine aldolase activity and collecting the compound of interest remaining in the reaction solution. Specifically, when the enzymatically active material reacts with the starting material DL-erythro-β-hydroxyamino acid, it stereoselectively cleaves the substrate L-erythro-β-hydroxyamino acid. As a result, the compound of interest, D-erythro-β-hydroxyamino acid, remains in the reaction solution. Alternatively, when the enzymatically active material reacts with the starting material DL-threo-α-hydroxyamino acid, it stereoselectively cleaves the substrate L-threo-β-hydroxyamino acid. As a result, the compound of interest, D-threo-β-hydroxyamino acid, remains in the reaction solution.

In the present invention, there is no limitation on the type of procedure for contacting the enzymatically active material and a reaction solution containing the starting material. For example, the enzymatically active material can be combined with a solvent containing the starting material to contact the two materials. When the enzymatically active material is insoluble in the solvent, the enzymatically active material may be dispersed in the solvent, and if required the two materials may be separated. Alternatively, the solvent containing the starting material can be contacted with the enzymatically active material while the two are being separated via a substrate-permeable membrane. Such a contact method facilitates the recovery and recycling of the enzymatically active material. In the present invention, the method for contacting the two materials is not limited to the specific examples indicated herein.

In the present invention, a transformant expressing the functional protein comprising the sequence of SEQ ID NO: 2 or a homologue thereof, and a processed product thereof, can be used as the enzymatically active material. For example, E. coli transformed with pKK-PSA1 or pSE-PSA1 is a preferred transformant in the present invention.

Specifically, a processed product of the transformant expressing the protein of SEQ ID NO: 2 or a homologue thereof according to the present invention includes the enzymatically active material listed below.
Microorganisms which have been treated with a detergent or an organic solvent, such as toluene, to change the cell membrane permeability;
Dried microbial cells prepared by freeze-drying or spray-drying;
Cell-free extract prepared by crushing microbial cells using glass beads or an enzyme;
Partially purified cell-free extract;
Purified enzyme; and
Immobilized enzyme and immobilization microorganism prepared by immobilizing a transformant or an enzyme.

The present invention provides methods of enzymatically producing optically active D-β-hydroxyamino acid from DL-erythro-α-hydroxyamino acid or DL-threo-β-hydroxyamino acid as starting material. D-β-hydroxyamino acid can be produced as the erythro isomer or threo isomer with high optical purity by the methods of the present invention. In other words, D-β-hydroxyamino acids with high optical purity can be produced more simply and at lower cost by using the methods of the present invention as compared with the conventional methods using optical resolving agents.

Furthermore, such a compound of interest can be obtained efficiently using a larger amount of starting material according to the present invention. More specifically, the present invention makes it possible to produce, for example, D-erythro-2-amino-3-cyclohexyl-3-hydroxypropionic acid simply and cost-effectively on an industrial scale from a practical amount of racemate as the starting material. In other words, the enzymatically active materials according to the present invention are hardly inhibited by a large amount of substrate and the reaction product generated from the substrate. Thus, the present invention makes it possible to efficiently produce a compound of interest using a larger amount of starting material.

All patents, patent applications, and publications cited herein are hereby incorporated by reference herein in their entirety.

### EXAMPLES

The present invention is illustrated in detail below with reference to Examples, but should not to be construed as being limited thereto.

### REFERENCE EXAMPLE 1

Culture of Pseudomonas putida biovar A24-1 strain

Pseudomonas putida biovar A24-1 strain was inoculated to peptone medium (pH 7.2) containing 1.0% peptone, 0.2% dipotassium monohydrogen phosphate, 0.2% monopotassium dihydrogen phosphate, 0.2% sodium chloride, 0.01% magnesium sulfate heptahydrate, and 0.01% yeast extract. The cells were grown by shaking culture at 30° C. for 12 hours. An aliquot of the culture was inoculated to peptone medium containing 0.2% DL-threo-phenyl serine. The bacterial cells were grown by shaking culture at 30° C. for 24 hours, and then harvested by centrifugation.

### REFERENCE EXAMPLE 2

### Purification of L-Phenylserine Aldolase

289 g of the cells prepared according to Reference example 1 was suspended in a bacterial cell lysis buffer containing 0.1 M TES-NaOH (pH 7.2), 2 mM ethylene diamine tetraacetic acid (EDTA), and 0.02% 2-mercaptoethanol. The cell suspension was treated with a sonicator to lyse the bacterial cells. The bacterial cell lysate was centrifuged, and the resulting supernatant fraction was dialyzed against buffer 1 containing 10 mM TES-NaOH (pH 7.2), 1 mM EDTA, 0.01% 2-mercaptoethanol, and 50 μM PLP to prepare cell-free extract.

Ammonium sulfate was added to the cell-free extract until it was 40%-saturated with ammonium sulfate. The resulting precipitate was removed by centrifugation. Ammonium sulfate was added to the supernatant until it was 60%-saturated with ammonium sulfate. The enzyme was recovered in the precipitated fraction by centrifugation.

The precipitated fraction was dissolved in buffer 1, and dialyzed against the same buffer. Then, the enzyme was adsorbed onto a DEAE-cellulose column (4.8×38 cm) equilibrated with buffer 1. A stepwise elution was performed using buffer 1, and buffer 1 containing 0.1 M, 0.15 M, 0.2 M, and 0.25 M potassium chloride. The enzyme was eluted with buffer 1 containing 0.15 M and 0.2 M potassium chloride. The active fractions were collected and dialyzed against buffer 2 containing 10 mM potassium phosphate (pH 7.2) and 0.01% 2-mercaptoethanol.

Then, the enzyme was adsorbed onto a hydroxyapatite column (1.7×18 cm) equilibrated with buffer 2. A stepwise elution was performed with 0.01 M, 0.02 M, 0.03 M, 0.05 M, and 0.1 M potassium phosphate buffer. The active fractions eluted with 0.01 M, 0.02 M, and 0.03 M phosphate buffer were collected, and concentrated. The enzyme solution was dialyzed against buffer 1.

The enzyme dialyzed was adsorbed onto a DEAE-cellulose column (1.7×19 cm) equilibrated with buffer 1. The column was washed with the same buffer, and then a stepwise elution was performed with buffer 1 containing 0.1 M, 0.15 M, 0.2 M, and 0.25 M potassium chloride. The enzyme was eluted with buffer 1 containing 0.15 M potassium chloride. The active fractions were concentrated, and then dialyzed against buffer 2.

The enzyme was adsorbed onto a hydroxyapatite column (1.7×18 cm) equilibrated with buffer 2. The column was washed with buffer 2, and then eluted with 0.01 M, 0.03 M, and 0.05 M potassium phosphate buffer. The enzyme was eluted at the concentration of 0.03 M potassium phosphate. The active fractions were collected and concentrated. The enzyme solution was dialyzed against buffer 1.

The enzyme was adsorbed onto a MonoQ column (0.5×5 cm) equilibrated with buffer 1. The column was washed with the same buffer, and then eluted with a gradient of 0 to 0.5 M potassium chloride. The eluted active fractions were collected as the purified enzyme.

The purified enzyme was fractionated by gel electrophoresis. The sample gave single bands in both native polyacrylamide and sodium dodecyl sulfate (SDS)-polyacrylamide gel electrophoreses.

The course of purification is summarized in Table 1.

TABLE 1
Purification of L-phenylserine aldolase

| | | | Specific | |
|---|---|---|---|---|
| | Total protein | Total activity | activity | Yield |
| Step | (mg) | (U) | (U/mg) | (fold) |
| cell-free extract | 38100 | 25500 | 0.669 | 1 |
| 40-60% ammonium | 24200 | 25400 | 1.05 | 1.57 |
| sulfate fraction | | | | |
| DEAE-cellulose (1st) | 8670 | 21300 | 2.46 | 3.67 |
| Hydroxyapatite (1st) | 803 | 8190 | 10.2 | 15.2 |
| DEAE-cellulose (2nd) | 98.7 | 4690 | 47.5 | 71.0 |
| Hydroxyapatite (2nd) | 29.2 | 2180 | 74.7 | 112 |
| MonoQ | 8.8 | 2730 | 310 | 464 |

### REFERENCE EXAMPLE 3

### Molecular Weight of L-Phenylserine Aldolase

The purified enzyme was fractionated by gel filtration using a TSK gel G3000 SW column (Tosoh) with an elution buffer containing 10 mM TES-NaOH (pH 7.2), 0.01% 2-mercaptoethanol, and 0.1 M potassium chloride. The result showed that the molecular weight was about 210,000. When the enzyme was fractionated by gel filtration using Sephadex G-200 with the same buffer, its molecular weight was estimated to be about 190,000.

The molecular weight of the subunit was estimated to be about 35,000 by SDS-polyacrylamide gel electrophoresis. The enzyme was predicted to be a homohexamer based on these results.

### REFERENCE EXAMPLE 4

### Optimal pH for L-Phenylserine Aldolase

The activity of L-phenylserine aldolase was assayed using sodium acetate buffer, TES-NaOH buffer, Tris-hydrochloride buffer, TAPS-NaOH buffer, and glycine-NaOH buffer. The activity of cleaving DL-threo-phenyl serine was evaluated and indicated in FIG. 1.

The optimal pH was achieved using TAPS-NaOH buffer (pH 8.5).

### REFERENCE EXAMPLE 5

### Substrate Specificity of L-Phenylserine Aldolase

The activity of L-phenylserine aldolase purified according to Reference example 2 was evaluated using various substrates under the standard assay conditions.

Table 2 shows the relative activities determined when the activity to DL-threo-β-phenyl serine is taken as 100.

TABLE 2
Substrate specificity of L-phenylserine aldolase

| Substrate | Relative activity |
|---|---|
| DL-threo-β-phenyl serine | 100 |
| DL-erythro-β-phenyl serine | 130 |
| L-threonine | 2.1 |
| D-threonine | 0 |
| L-allo-threonine | 8 |
| D-allo-threonine | 0 |
| L-serine | 0 |
| D-serine | 0 |
| DL-β-thienyl serine | 170 |
| DL-β-hydroxynorvaline | 0 |
| L-phenyllactic acid | 0 |
| DL-β-hydroxyphenylethylamine | 0 |

### REFERENCE EXAMPLE 6

### Effect of Cations on the Activity of L-Phenylserine Aldolase

The activity of L-phenylserine aldolase was assayed under the standard assay conditions. The enzyme and a cation were added to the reaction solution without the substrate DL-threo-β-phenyl serine and PLP. The mixture was incubated at 30° C. for 10 minutes. Then, DL-threo-α-phenyl serine and PLP were added to the mixture to assay the enzymatic activity. Table 3 shows the relative activity determined when the activity in the absence of cation is taken as 100.

TABLE 3
Effect of cations on the activity of L-phenylserine aldolase

| | Concentration | Relative activity |
|---|---|---|
| Compound | (mM) | (%) |
| Without cation | — | 100 |
| Potassium chloride | 50 | 94 |
| Ammonium chloride | 50 | 92 |
| Sodium chloride | 50 | 88 |
| Aluminum sulfate | 0.1 | 92 |
| Barium chloride | 0.1 | 90 |
| Manganese chloride | 0.1 | 92 |
| Copper sulfate | 0.1 | 89 |
| Nickel sulfate | 0.1 | 91 |
| Cobalt chloride | 0.1 | 98 |
| Magnesium chloride | 0.1 | 98 |
| Ferric (II) sulfate | 0.1 | 91 |
| Zinc chloride | 0.1 | 92 |

### REFERENCE EXAMPLE 7

### Method for Producing 3-cyclohexyl-2-propenoic Acid

A mixture of malonic acid (23.9 g) and toluene (58.1 g) was heated to 65° C. 27.1 g of pyridine and 0.54 g of piperidine were added dropwise to the mixture. Then, 30.0 g of cyclohexyl aldehyde was added dropwise and allowed to react for 10 hours. After reaction, the mixture was cooled, and 93.3 g of an aqueous solution of 3 M sodium hydroxide was added to the mixture. After stirring, toluene of the top layer was discarded. 55.5 g of t-butylmethyl ether and 55.8 g of concentrated hydrochloric acid were added to the bottom layer. After stirring, the bottom layer was discarded. 24.8 g of water and 0.29 g of concentrated hydrochloric acid were added to t-butyl methyl ether of the top layer. After stirring, the bottom layer was discarded. t-butyl methyl ether of the top layer was concentrated under reduced pressure to give 33.0 g of 3-cyclohexyl-2-propenoic acid.

### REFERENCE EXAMPLE 8

### Production of 3-cyclohexyl-2,3-epoxy propionic acid

4.3 g of sodium tungstate dihydrate was dissolved in 64.4 g of water. The mixture was heated to 40° C. 20 g of 3-cyclohexyl-2-propenoic acid and 15.8 g of methanol were added to the mixture. Then, 29.4 g of 30% hydrogen peroxide solution was added dropwise to the mixture. The resulting mixture was incubated at 40° C. for 20 hours, while the pH of the reaction solution was being controlled to fall within the range of 4.5 to 5.0 using an aqueous solution of 25% sodium hydroxide. After reaction, the solution was cooled to 10° C. or lower temperatures. Hydrogen peroxide was decomposed by adding dropwise an aqueous solution of 35% sodium bisulfite and an aqueous solution of 25% sodium hydroxide while the pH of the solution is controlled to fall within the range of 4.0 to 6.0. After methanol was distilled off under reduced pressure, the pH was adjusted to 2.0 to 3.0 with concentrated hydrochloric acid. 676 g of t-butyl methyl ether was added, and the resulting mixture was stirred for 30 minutes or longer period. Then, the mixture was allowed to stand still for 30 minutes or longer period. The bottom layer was discarded, and then the top layer was treated by filtration. The filtrate was concentrated under reduced pressure to give 16.2 g of pale yellow, oily 3-cyclohexyl-2,3-epoxy propionic acid.

### REFERENCE EXAMPLE 9

### Production of 2-benzylamino-3-cyclohexyl-3-hydroxypropionic acid

10.0 g of 3-cyclohexyl-2,3-epoxy propionic acid was dissolved in a mixture of 21.0 g of water and 9.4 g of an aqueous solution of 25% sodium hydroxide. The resulting mixture was heated to 40 to 55° C. 18.9 g of benzylamine was added dropwise and allowed to react at 90° C. for ten hours. After reaction, the solution was cooled to 30° C. or lower temperatures. The reaction solution was added dropwise to a mixture of 17.1 g of concentrated hydrochloric acid, 61.1 g of water, and 9.9 g of t-butyl methyl ether. Then, the pH of the resulting mixture was adjusted to 3.5 using an aqueous solution of 25% sodium hydroxide. The resulting mixture was stirred for 30 minutes. The precipitated crystals were collected by filtration. The crystals were washed with acetone and with water, and then dried under reduced pressure at 50° C. to give 13.9 g of 2-benzylamino-3-cyclohexyl-3-hydroxypropionic acid.

### REFERENCE EXAMPLE 10

### Method for Producing 2-amino-3-cyclohexyl-3-hydroxypropionic acid

62.8 g of methanol, 39.4 g of water, 7.5 g of an aqueous solution of 25% sodium hydroxide, and as a catalyst 0.7 g of palladium hydroxide (supported on activated carbon) containing 50% water were added to 13.0 g of 2-benzylamino-3-cyclohexyl-3-hydroxypropionic acid. The mixture was reacted under a pressure of 500 KPa with hydrogen gas at 50° C. for 4 hours. After reaction, 7.7 g of an aqueous solution of 12% sodium hydroxide was added, and the resulting mixture was stirred for 30 minutes. The catalyst was removed by filtration. The filtrate was concentrated under reduced pressure to remove methanol and toluene. The pH of the solution was adjusted to 5.8 using concentrated hydrochloric acid. The precipitated crystals were collected by filtration, and washed with water. The crystals were dried under reduced pressure to give 7.9 g of 2-amino-3-cyclohexyl-3-hydroxypropionic acid.

### EXAMPLE 1

### Purification of Chromosomal DNA from Pseudomonas putida

Pseudomonas putida biovar A24-1 strain was cultured in a medium containing 1.5% polypeptone, 0.5% yeast extract, and 0.5% sodium chloride, and then the bacterial cells were harvested. The chromosomal DNA was purified from the bacterial cells by the method described in Biochem. Biophys. Acta., 72, 619 (1963).

### EXAMPLE 2

### Cloning of L-Phenylserine Aldolase Gene Core Sequence

The enzyme purified in Reference example 2 was digested with trypsin. Primer 12 and primer C were synthesized based on the amino acid sequences of the peptides obtained (SEQ ID NOs: 3 and 4).

SEQ ID NO: 3

Gln-Ala-Gly-Pro-Tyr-Gly-Thr-Asp-Glu-Leu

SEQ ID NO: 4

Phe-Gly-Phe-Tyr-His-Asp-Arg-Trp

SEQ ID NO: 5

primer 12: GGGAATTCAGGCGGGCCCGTATGGCACCGACCGACGA

SEQ ID NO: 6

primer C: AAGCCGAAGATAGTGCTGGGCGACCCCTAGGGG

PCR was carried out using a DNA Thermal Cycler (Perkin Elmer) and 50 μL of the reaction solution containing the pair of primers (50 pmol each), 10 nmol dNTP, 50 ng of chromosomal DNA, AmpliTaq DNA polymerase buffer (Perkin Elmer), 3 mM MgCl₂, and 2.5 U AmpliTaq DNA polymerase (Perkin Elmer). The thermal cycling profile consists of: 30 cycles of denaturation (94° C., 1 minute), annealing (50° C., 2 minutes), and extension (65° C., 3 minutes).

The nucleotide sequence of the amplified DNA was determined by performing PCR using BigDye Terminator Cycle Sequencing FS ready Reaction Kit (Perkin Elmer) in a DNA sequencer ABI PRISM™ 310 (Perkin Elmer). The sequence obtained is shown in SEQ ID NO: 7.

### EXAMPLE 3

### Cloning of the Entire L-phenylserine Aldolase Gene

Two oligonucleotides, probe U2 and probe C, were synthesized based on the nucleotide sequence of the core sequence. Southern hybridization was carried out using the synthesized DNAs as probes.

SEQ ID NO: 8

probe U2: TGATGACCGTCGACGGCCCG

SEQ ID NO: 9

probe C: CGGCCTTCAGCAGCGCATCGA

Specifically, chromosomal DNA was digested with the restriction enzyme SphI. The digested DNA was fractionated by agarose gel electrophoresis, and then transferred onto a nylon membrane (Du Pont). After the membrane was washed with 2×SSC, the DNA was alkali-immobilized with 0.4 M NaOH for 1 minute. Then, the membrane was air-dried after neutralization with 0.2 M Tris-hydrochloride buffer (pH 7.5) and 2×SSC. The membrane was pre-hybridized in a hybridization solution (0.1% bovine serum albumin, 0.1% Ficoll, 0.1% polyvinylpyrrolidone, 0.75 mM NaCl, 75 mM sodium citrate, and 1% SDS) at 45-55° C. overnight. Then, the membrane was hybridized to the ³²P-labeled probes in the hybridization solution containing 250 μg of heat-denatured salmon sperm DNA at 45-55° C. overnight. After hybridization, the membrane was washed with a solution containing 6×SSC and 0.5% SDS. Then, the membrane was washed with the same solution at 55° C. for 2 minutes and shortly rinsed with 2×SSC. After the membrane was air-dried, the signals on the membrane were visualized by autoradiography.

Both probes were confirmed to strongly hybridize to DNA fragment of about 6.7 kb obtained by digesting chromosomal DNA with SphI. Then, the DNA fragment was extracted from the gel and ligated using TaKaRa DNA Ligation Kit (Takara Bio) into pUC19 digested with the same enzyme SphI. E. coli JM109 strain was transformed with the ligated DNA. About 700 clones were yielded by the transformation.

The approximately 700 clones were screened by colony hybridization using the synthetic DNA probe U2. As a result, 41 colonies exhibiting stronger radioactivity were selected. A plasmid containing about 6.7-kb DNA fragment of interest as an insert was selected from the clones. The plasmid was named pPSA2.

The nucleotide sequence of the DNA fragment inserted in pPSA2 was analyzed, and the result showed that the fragment contained the 1074-bp open reading frame (ORF) containing the core sequence. The determined nucleotide sequence and the amino acid sequence deduced from the nucleotide sequence are shown in SEQ ID NOs: 1 and 2, respectively.

### EXAMPLE 4

### Construction of Overexpression Plasmid for the L-Phenylserine Aldolase Gene, pKK-PSA1

A strain expressing the L-phenylserine aldolase at high levels was established. First, oligonucleotide primers PSA-ECO and PSA-HIN, which were placed upstream and downstream of the ORF of the L-phenylserine aldolase gene, were synthesized based on the nucleotide sequence of the DNA fragment inserted in pPSA2.

SEQ ID NO: 10

primer PSA-ECO: GGGAATTCGACCATCAGGCGAGCGTCAA

SEQ ID NO: 11

primer PSA-HIN: GGAAGCTTCCAGAGCGAGCACAGCCGCCAC

PCR was carried out using 50 μl of the reaction solution containing the primers (10 pmol each), 0.2 mM dNTP, AmpliTaq DNA polymerase buffer (Perkin Elmer), 2.5 U AmpliTaq DNA polymerase (Perkin Elmer), and the plasmid pPSA2 as a template, and a DNA Thermal Cycler (Perkin Elmer). The thermal cycling profile used consisted of 30 cycles of denaturation (94° C., 30 seconds), annealing (50° C., 30 seconds), and extension (72° C., 1 minute). The amplified DNA fragment was double-digested with the restriction enzymes EcoRI and HindIII. pKK-PSA1 was obtained by ligating the digested fragment with pKK223-3 which had been double-digested with the same restriction enzymes. The plasmid pKK-PSA1 containing the L-phenylserine aldolase gene was deposited under the accession number ______. The deposited material can be identified based on the information indicated below.
(a) Name and Address of Depositary Institution
   Name: International Patent Organism Depositary, National Institute of Advanced Industrial Science and Technology (AIST), Independent Administrative Institution
   Address: AIST Tsukuba Central 6, 1-1-3 Higashi, Tsukuba, Ibaraki, Japan (Zip Code: 305-8566)
(b) Date of Deposition: ______
(c) Accession No: ______

### EXAMPLE 5

### Test for the Activity of L-Phenylserine Aldolase

E. coli JM109 strain was transformed with the plasmid pKK-PSA1 obtained in Example 4. The resulting transformant was inoculated to liquid LB medium (Bacto-tryptone 10 g/l, Bacto-yeast extract 5 g/l, sodium chloride 10 g/l; pH 7.2) containing ampicillin (50 mg/l), and cultured at 30° C. overnight. Then, an aliquot of the culture was inoculated to 2×YT medium (Bacto-tryptone 20 g/l, Bacto-yeast extract 10 g/l, sodium chloride 10 g/l; pH 7.2) containing ampicillin (50 mg/l). The bacterial cells were cultured at 30° C., and then the induction was achieved by treating the cells with 0.1 mM IPTG for 4 hours. The bacterial cells were harvested by centrifugation. The cells were suspended in 100 mM TES-NaOH buffer (pH 7.2) containing 0.02% 2-mercaptoethanol, 2 mM EDTA, and 20 μM pyridoxal-5′-phosphate. The cell suspension was sonicated in a closed sonicator UCD-200™ (Cosmo Bio) for 3 minutes to lyse the bacterial cells. The bacterial cell lysate was centrifuged and the resulting supernatant was used as cell-free extract. The activity of L-phenylserine aldolase was assayed using the cell-free extract. The specific activity was confirmed to be 20.0 U/mg.

### EXAMPLE 6

### The activity of the L-phenylserine aldolase to DL-erythro-2-amino-3-cyclohexyl-3-hydroxypropionic acid

The cell-free extract obtained in Example 5 was assayed for the enzymatic activity to DL-erythro-2-amino-3-cyclohexyl-3-hydroxypropionic acid. The activity was found to be 6.26 U/mg. When the activity to DL-threo-phenyl serine was taken as 100%, the relative activity was estimated to be 31.3%.

### EXAMPLE 7

### Synthesis of D-erythro-2-amino-3-cyclohexyl-3-hydroxypropionic acid using E. coli JM109 (pKK-PSA1)

D-erythro-2-amino-3-cyclohexyl-3-hydroxypropionic acid was synthesized using E. coli JM109 strain containing the plasmid pKK-PSA1 obtained in Example 6.

10 ml of a reaction solution containing 400 mM Tris-HCl buffer (pH 8.5), 50 μM pyridoxal-5′-phosphate, DL-erythro-2-amino-3-cyclohexyl-3-hydroxypropionic acid, and the bacterial cells was reacted under agitation at 30° C. overnight. After reaction, remaining D-erythro-2-amino-3-cyclohexyl-3-hydroxypropionic acid was quantified and its optical purity was determined by the procedure described below.

After the reaction was terminated, the solution was diluted with 1N HCl, and centrifuged. The resulting supernatant was analyzed using CROWNPAK CR(+) (0.46×15 cm; Daicel Chemical Industries, Ltd.). D-erythro isomer was eluted after 24.2 minutes, while L-erythro isomer was eluted after 28.0 minutes.

The results are shown in Table 4. The unnecessary isomer completely cleaved at any DL-erythro-2-amino-3-cyclohexyl-3-hydroxypropionic acid concentrations of 1%, 5%, 10%, and 15%. The remaining D-erythro-2-amino-3-cyclohexyl-3-hydroxypropionic acid was confirmed to give 99% e.e. or higher optical purity.

TABLE 4

| Substrate | |
|---|---|
| DL-erythro-2-amino-3-cyclohexyl- | D-erythro isomer |
| 3-hydroxypropionic acid | Optical purity |
| 1% | 99% e.e |
| 5% | 99% e.e |
| 10% | 99% e.e |
| 15% | 99% e.e |

### EXAMPLE 8

Purification of D-erythro-2-amino-3-cyclohexyl-3-hydroxypropionic acid (1)

After reaction, sulfuric acid was added to the solution reacted in the presence of 15% substrate in Example 7. D-erythro-2-amino-3-cyclohexyl-3-hydroxypropionic acid remaining in the reaction solution was dissolved by lowering the pH of the solution to 0.5. After removal of unnecessary material such as bacterial cells by centrifugation, sodium hydroxide was added to the resulting supernatant. D-erythro-2-amino-3-cyclohexyl-3-hydroxypropionic acid was crystallized by neutralizing the solution to about pH 6. The crystals were collected by filtration. The purity of crystals was about 96%, and the optical purity was >99% ee.

### EXAMPLE 9

### Purification of D-erythro-2-amino-3-cyclohexyl-3-hydroxypropionic acid (2)

After reaction, sodium hydroxide was added to the solution reacted in the presence of 15% substrate in Example 7. D-erythro-2-amino-3-cyclohexyl-3-hydroxypropionic acid remaining in the reaction solution was dissolved by increasing the pH of the solution to 12. After removal of unnecessary material such as bacterial cells by centrifugation, sulfuric acid was added to the resulting supernatant. D-erythro-2-amino-3-cyclohexyl-3-hydroxypropionic acid was crystallized by neutralizing the solution to about pH 6. The crystals were collected by filtration. The optical purity of the crystals was >99% ee.

### Industrial Applicability

The present invention provides methods for efficiently producing high yields of D-β-hydroxyamino acids, namely D-erythro-β-hydroxyamino acids and D-threo-β-hydroxyamino acids. D-β-hydroxyamino acids produced according to the present invention are useful as intermediates in the synthesis of pharmaceutical products or pesticides. More specifically, for example, D-erythro-2-amino-3-cyclohexyl-3-hydroxypropionic acid can be synthesized efficiently on an industrial scale according to the present invention.

While the invention has been described in detail and with reference to specific embodiments thereof, it will be apparent to one skilled in the art that various changes and modifications can be made therein without departing from the spirit and scope of the invention.

What is claimed is:

1. A method for producing D-erythro-β-hydroxyamino acid, which comprises the step of collecting D-erythro-β-hydroxyamino acid represented by formula 2 after DL-erythro-β-hydroxyamino acid represented by formula 1 (where R represents an optionally substituted cyclohexyl group, a phenyl group, an alkyl group, or an allyl group) 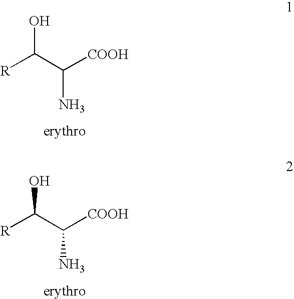
is reacted with at least one enzymatically active material selected from the group consisting of: a protein encoded by any one of the polynucleotides defined in (a) to (e) indicated below, a microorganism or transformant expressing the protein, and a processed product thereof;
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added, further wherein the resulting protein is functionally equivalent to the protein comprising the amino acid sequence of SEQ ID NO: 2;
(d) a polynucleotide hybridizing under stringent conditions to a DNA comprising the nucleotide sequence of SEQ ID NO: 1, wherein said polynucleotide encodes a protein that is functionally equivalent to the protein comprising the amino acid sequence of SEQ ID NO: 2; and
(e) a polynucleotide encoding an amino acid sequence having 70% or higher homology to the amino acid sequence of SEQ ID NO: 2, wherein said polynucleotide encodes a protein that is functionally equivalent to the protein comprising the amino acid sequence of SEQ ID NO: 2.

2. The method for producing D-erythro-β-hydroxyamino acid according to claim 1, wherein R is an optionally substituted cyclohexyl group.

3. A method for producing D-threo-β-hydroxyamino acid, which comprises the step of collecting D-threo-β-hydroxyamino acid represented by formula 4 after DL-threo-β-hydroxyamino acid represented by formula 3 (where R represents an optionally substituted cyclohexyl group, a phenyl group, an alkyl group, or an allyl group) 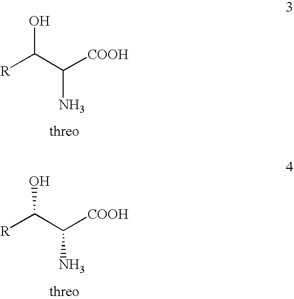
is reacted with at least one enzymatically active material selected from the group consisting of a protein encoded by any one of the polynucleotides defined in (a) to (e) indicated below, a microorganism or transformant expressing the protein, and a processed product thereof,
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added, further wherein the resulting protein is functionally equivalent to the protein comprising the amino acid sequence of SEQ ID NO: 2;
(d) a polynucleotide hybridizing under stringent conditions to a DNA comprising the nucleotide sequence of SEQ ID NO: 1, wherein said polynucleotide encodes a protein that is functionally equivalent to the protein comprising the amino acid sequence of SEQ ID NO: 2; and
(e) a polynucleotide encoding an amino acid sequence having 70% or higher homology to the amino acid sequence of SEQ ID NO: 2, wherein said polynucleotide encodes a protein that is functionally equivalent to the protein comprising the amino acid sequence of SEQ ID NO: 2.

4. The method for producing D-threo-α-hydroxyamino acid according to claim 3, wherein R is an optionally substituted cyclohexyl group.

5. The method for producing D-β-hydroxyamino acid according to claim 1 or 3, wherein the concentration of material DL-β-hydroxyamino acid is 30 g/l or higher in the reaction solution.

6. The method for producing D-β-hydroxyamino acid according to claim 1 or 3, wherein the concentration of material DL-β-hydroxyamino acid is 50 g/l or higher in the reaction solution.

7. The method for producing D-erythro-α-hydroxyamino acid or D-threo-β-hydroxyamino acid according to claim 1 or 3, wherein the concentration of material DL-erythro-β-hydroxyamino acid or DL-threo-β-hydroxyamino acid is 50 g/l or higher in the reaction solution.

8. The method for producing D-erythro-β-hydroxyamino acid or D-threo-β-hydroxyamino acid according to claim 2 or 4, which additionally comprises the steps of:
(1) dissolving D-erythro-α-hydroxyamino acid or D-threo-β-hydroxyamino acid by adjusting the pH of the reaction solution to 10 or higher after the reaction;
(2) separating insoluble materials, and
(3) collecting D-erythro-β-hydroxyamino acid or D-threo-β-hydroxyamino acid precipitated by adjusting the pH of the reaction solution to 2 to 9.5.

9. The method for producing D-erythro-α-hydroxyamino acid or D-threo-α-hydroxyamino acid according to claim 2 or 4, which additionally comprises the steps of:
(1) dissolving D-erythro-β-hydroxyamino acid or D-threo-β-hydroxyamino acid by adjusting the pH of the reaction solution to 1.5 or lower after the reaction;
(2) separating insoluble materials, and
(3) collecting D-erythro-β-hydroxyamino acid or D-threo-β-hydroxyamino acid precipitated by adjusting the pH to 2 to 9.5.
